(19) United States
(12) Patent Application Publication
(10) Pub. No.: US 2016/0250481 A1
(43) Pub. Date: Sep. 1, 2016

(54) MAGNETIC FIELD SENSOR ARRANGEMENT

(71) Applicant: BIOTRONIK SE & Co. KG, Berlin (DE)

(72) Inventors: James W. Hess, Aloha, OR (US); Richard Berthelsdorf, Dundee, OR (US)

(21) Appl. No.: 15/001,342

(22) Filed: Jan. 20, 2016

Related U.S. Application Data
(60) Provisional application No. 62/120,909, filed on Feb. 26, 2015.

(51) Int. Cl. A61N 1/37; A61N 1/08; G01R 33/09

(52) U.S. Cl. CPC A61N 1/3718; G01R 33/093; A61N 1/08

(57) ABSTRACT

A magnetic field sensor includes at least one sensing circuit including giant magnetoresistive (GMR) elements. At least one of the GMR elements is at least partially housed in a flux-reducing or flux-concentrating first shielding material that reduces or amplifies, respectively, the magnetic field impinging on the element(s), while at least one other GMR element is housed in a second shielding material having an effect opposite that of the first shielding material, or is not housed in any shielding material. A measurement circuit is configured to measure voltage between different points of the GMR sensing circuit, allowing derivation of electric impedance between the different measurement points. An evaluation circuit evaluates the impedances and the relationships therebetween to derive a signal that indicates the strength of an applied magnetic field.

### CROSS-REFERENCE TO RELATED APPLICATIONS

This application claims priority under 35 USC §119(e) to U.S. Provisional Patent Application 62/120,909 filed 26 Feb. 2015 the entirety of which is incorporated by reference herein.

### FIELD OF THE INVENTION

The invention relates to a magnetic field sensor arrangement for sensing magnetic fields of different strengths. In particular, the invention relates to an implantable medical device, e.g. an implantable pulse generator such as a heart stimulator or a neurostimulator, including a magnetic field sensor arrangement for sensing magnetic fields of different strengths. The invention also relates to a method for determining the presence of a magnetic field.

### BACKGROUND OF THE INVENTION

For more than 45 years, manufacturers have used a reed switch in cardiac implants to set the implant into a mode of operation that is commonly referred to as the magnet mode. The reed switch is normally open, and is closed when a permanent magnet is brought into close proximity to the implant.

Magnetic Resonance Imaging (MRI) is a popular diagnostic tool which is not recommended for use with pacemaker and implantable cardioverter/defibrillator (ICD) patients because its magnetic fields can induce mechanical forces on the implant, heating of implant leads, permanent damage to the implant's electronic circuit, or implant therapy malfunction (e.g. loss of sensing or pacing at the upper tracking rate (UTR)). MRI machines typically use a static magnetic field with field strength of 1.5T, although fields of 3T or higher may be present in newer machines. A reed switch is unable to differentiate between different magnetic field strength levels, and when exposed to a MRI or other magnetic field, it will remain closed, placing the device into its magnet mode.

As an alternative to reed switches, a Hall effect sensor can be used to detect magnetic fields. A Hall effect sensor has the advantage that its output voltage is proportional to the strength of the magnetic field, enabling determination of whether the magnetic field is from a normal permanent magnet or from an MRI. U.S. Pat. No. 6,937,906 describes an implantable pacemaker able to detect an MRI magnetic field, and automatically change the sensing mode of the device to one less affected by the MRI signal.

A GMR (Giant MagnetoResistance) sensor is also a known alternative to a reed switch, as seen in U.S. Pat. No. 6,101,417, U.S. Patent Appl'n. Publ'n. 2008/0154342, and US Patent Appl'n. Publ'n. 2011/0202104. A GMR's response to a magnetic field varies as the angle of the field changes relative to the GMR. The use of multiple GMRs mounted at various angles to each other can compensate for this, but using multiple GMRs results in more components, higher cost, and higher current consumption.

### SUMMARY OF EXEMPLARY VERSIONS OF THE INVENTION

The invention seeks to provide a magnetic field sensor arrangement that allows reliable detection of magnetic fields of different strengths and orientations while keeping costs and energy consumption low. A magnetic field sensor arrangement of this arrangement may include at least one GMR sensing circuit including GMR elements. At least one of the GMR elements is at least partially housed in a flux-reducing or flux-concentrating shielding material that reduces or amplifies, respectively, the magnetic field impinging on it, while at least one other GMR element is not housed in such material, or is housed in a material having an opposite effect than the shielding material. The magnetic field sensor further includes a measurement circuit configured for voltage measurement at different points of the GMR sensing circuit for deriving the electric impedance between the different measurement points, thereby deriving a signal that indicates a level of an external magnetic field.

In a preferred version of the invention, the magnetic field sensor arrangement further includes an evaluation circuit configured to evaluate the measured impedances and the relations of the measured impedances to derive a signal that indicates a level of an external magnetic field.

Replacing a reed switch with a GMR, and using that same GMR for MRI detection, allows a feature to be added to the implant while lowering the component cost of the device. In addition, the deletion of a mechanical element—the reed switch—enhances the reliability of the implant.

By combining the use of shielded and non-shielded GMR elements along with using multiple GMRs oriented at various angles relative to each other, the GMR's sensitivity to its orientation in relation to the magnetic field can be reduced, thus providing better field detection and reducing the costs of the device.

By monitoring the overall resistance change of a GMR sensor circuit in a bridge configuration (e.g., in a Wheatstone bridge), higher-level fields, such as those present during MRI, may also be detected using the same device. This can result in the use of fewer GMR components, thus lowering the cost of the field sensor.

The GMR-based magnetic field sensor of the invention allows more reliable MR detection, MRI detection in implants such as pacemakers, and reduction of the area required on the circuit board. Replacing the reed switch with a GMR also allows cost reduction.

Using different connections to a single GMR bridge, and thus allowing different measurement points with use of only one component, assists in keeping costs down and limiting current consumption (and thus increasing battery life).

The invention measures a resistance change of a combination of shielded and unshielded GMR elements. The term “shielded” refers to GMR elements having shields that reduce or concentrate the magnetic field impinging on them. The invention allows a good balance to be obtained between magnetic field sensitivity in the least sensitive GMR element to the orientation of a magnetic field having some upper field strength (e.g., 30 mT), and not detecting fields below some lower value (e.g., 1 mT to 10 mT) in the most sensitive orientation.

At least one GMR sensing circuit is preferably a bridge circuit including four GMR elements in a full bridge (Wheatstone bridge) configuration. The full bridge is formed with four GMR elements. Two of the legs, opposite each other, are shielded. The other two legs are not shielded. The shields result in a somewhat different response of the GMR to a magnetic field over a specific range. This difference results in a differential output that can be used to detect or measure the magnetic field.

The magnetic field sensor arrangement allows removal of a reed switch and addition of an MRI detection function with one component instead of two components, thereby providing added functionality (MRI detection) with no component cost increase.

The GMR elements are preferably resistors implemented with giant magneto-resistive material (GMR material).

The magnetic field sensor arrangement preferably includes at least two GMR sensing circuits which include GMR elements with different spatial orientations. A GMR has a strong sensitivity to magnetic fields aligned with its axis of sensitivity, and a weak sensitivity to fields that are orthogonal to the GMR's axis of sensitivity. MRI detection in implants requires the ability to sense a field at any angle while keeping the number of required components and cost to a minimum.

The invention also advantageously avoids problems clue to saturation effects of GMR bridges with shielded and non-shielded GMR sensors. For example, such problems can manifest in such a way that the differential output of a GMR bridge indicates the presence of a high magnetic field when exposed to a low magnetic field, or indicates the presence of no magnetic field when exposed to a high magnetic field, when exposed to changing magnetic field strengths.

In a configuration with GMR elements arranged at different angles, it is preferred that at least one GMR element has an orientation that differs from the orientation of at least one other GMR element by an oblique angle, i.e., a non-perpendicular angle of more than 0°.

Preferably, the GMR elements are situated in a common plane.

The magnetic field sensor arrangement preferably includes a shorting means for shorting at least one of the GMR elements, allowing easy generation of a further measurement point for voltage measurement. The shorting means preferably include at least one switch to short a GMR element.

The evaluation circuit is preferably configured to evaluate the relationships within the set of measured impedances by determining the differences of the measured impedances.

The invention also involves an implantable medical device including a magnetic field sensor arrangement as described in this document. Preferably, the implantable medical device is programmable and includes a processor that is operationally connected to the magnetic field sensor arrangement. In particular, the implantable medical device can be an is implantable pulse generator for generating stimulation pulses.

The invention also involves a method for determining the presence of a magnetic field, the method including: provision of a magnetic field sensor arrangement which includes a GMR sensing circuit as described in this document; measurement of the voltages between different points of the GMR sensing circuit; evaluation of the measured impedances and the relations of the measured impedances; and derivation of a signal that indicates the level of an applied magnetic field, as determined using the measured impedances and the relations therebetween.

### BRIEF DESCRIPTION OF THE DRAWINGS

FIG. 1 shows a dual chamber pacemaker connected to leads placed in a heart.

FIG. 2 shows a block diagram of a heart stimulator including a magnetic field sensor arrangement according to the invention.

FIG. 3 shows a GMR sensing circuit.

FIGS. 4-8 illustrate measurement configurations that allow further voltage measurements on a GMR sensing circuit similar to the one depicted in FIG. 3.

FIGS. 9-15 illustrate magnetic field sensor arrangements including two GMR sensing circuits.

FIGS. 16-22 illustrate versions wherein the magnetic field sensor arrangement includes three GMR sensing circuits.

FIG. 23 illustrates a version of a magnetic field sensor arrangement including four GMR sensing circuits.

### DETAILED DESCRIPTION OF EXEMPLARY VERSIONS OF THE INVENTION

The accompanying drawings and the following discussion merely relate to exemplary is versions of the invention, particularly an exemplary version where the implantable device is a cardiac pacemaker. The true scope of the invention should be determined with reference to the claims, which define the versions of the invention encompassed by this patent.

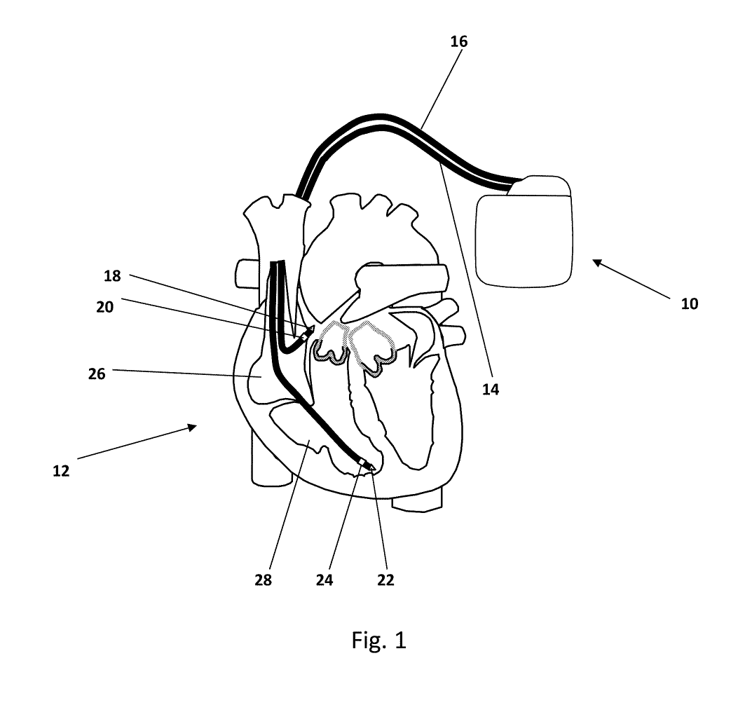

In FIG. 1, a heart stimulator, more specifically a dual chamber pacemaker 10, is connected to pacing/sensing leads placed in a heart 12. The pacemaker 10 is electrically coupled to the heart 12 by way of leads 14 and 16. The lead 14 has a pair of right atrial electrodes 18 and 20 in contact with the right atrium 26 of the heart 12. Lead 16 has a pair of electrodes 22 and 24 in contact with the right ventricle 28 of the heart 12. Electrodes 18 and 22 are tip electrodes at the distal ends of leads 14 and 16, respectively. Electrode 18 is a right atrial tip electrode (often denoted RA-Tip) and electrode 22 is a right ventricular tip electrode (RV-Tip). Electrodes 20 and 24 are ring electrodes in close proximity to, but electrically isolated from, the respective tip electrodes 18 and 22. Electrode 20 forms a right atrial ring electrode (RA-Ring) and electrode 24 forms a right ventricular ring electrode (RV-Ring).

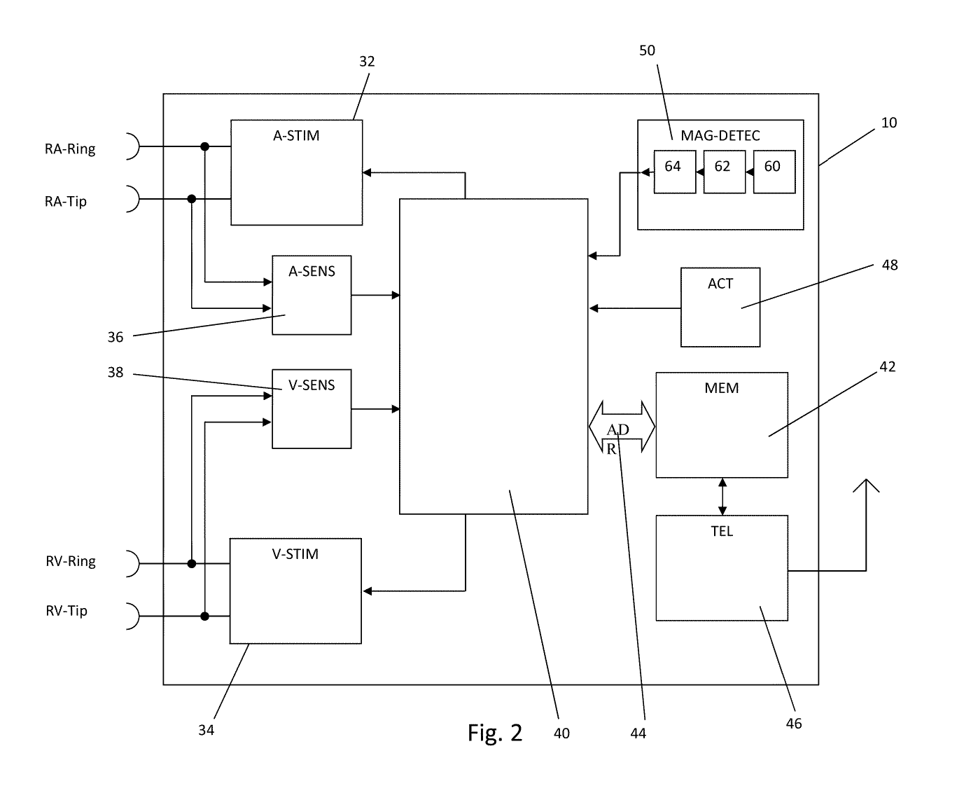

FIG. 2 provides a simplified block diagram of a dual chamber pacemaker 10. During operation of the pacemaker 10, leads 14 and 16 are connected to the respective output/input terminals of pacemaker 10 as indicated in FIG. 1, and carry stimulating pulses to the tip electrodes 18 and 22 from an atrial stimulation pulse generator A-STIM 32 and a ventricular pulse generator V-STIM 34, respectively. Further, electrical signals from the atrium are carried through lead 14 from the electrode pair 18 and 20 to the input terminal of an atrial channel sensing stage A-SENS 36; and electrical signals from the ventricles are carried through the lead 16 from the electrode pair 22 and 24 to the input terminal of a ventricular sensing stage V-SENS 38.

A control unit CTRL 40, connected to sensing stages A-SENS 36 and V-SENS 38 and to stimulation pulse generators A-STIM 32 and V-STIM 34, controls the dual chamber pacemaker 10. Control unit CTRL 40 receives the output signals from the atrial sensing stage A-SENS 36 and the ventricular sensing stage V-SENS 38. The output signals of sensing stages A-SENS 36 and V-SENS 38 are generated each time that a P-wave representing an intrinsic atrial event or an R-wave representing an intrinsic ventricular is event, respectively, is sensed within the heart 12. An As-signal is generated when the atrial sensing stage A-SENS 36 detects an atrial P-wave, and a Vs-signal is generated when the ventricular sensing stage V-SENS 38 detects a ventricular R-wave.

Control unit CTRL 40 also generates trigger signals that are sent to the atrial stimulation pulse generator A-STIM 32 and the ventricular stimulation pulse generator V-STIM 34, respectively. These trigger signals are generated each time that a stimulation pulse is to be generated by the respective pulse generator A-STIM 32 or V-STIM 34. The atrial trigger signal is referred to simply as the “A-pulse”, and the ventricular trigger signal is referred to as the “V-pulse”. During the time that either an atrial stimulation pulse or ventricular stimulation pulse is being delivered to the heart, the corresponding sensing stage, A-SENS 36 and/or V-SENS 38, is typically disabled by way of a blanking signal presented to these respective amplifiers from the control unit CTRL 40. This blanking action prevents the sensing stages A-SENS 36 and V-SENS 38 from becoming saturated from the relatively large stimulation pulses present at their input terminals during this time. This blanking action also helps prevent residual electrical signals present in the muscle tissue as a result of the pacer stimulation from being interpreted as P-waves or R-waves.

Furthermore, atrial sense events As recorded shortly after delivery of ventricular stimulation pulses during a preset time interval called the post ventricular atrial refractory period (PVARP) are generally recorded as atrial refractory sense events Ars, but ignored.

Control unit CTRL 40 includes circuitry for timing ventricular and/or atrial stimulation pulses according to a stimulation rate that can be adapted to a patient's hemodynamic need as discussed below.

Still referring to FIG. 2, the pacemaker 10 includes a memory circuit MEM 42 coupled to the control unit CTRL 40 over a suitable data/address bus ADR 44. This memory circuit

MEM 42 allows certain control parameters, used by the control unit CTRL 40 in controlling the operation of the pacemaker 10, to be programmably stored and modified as required in order to customize the pacemaker's operation to suit the needs of a particular patient. Such data includes the basic timing intervals used during operation of the is pacemaker 10.

Further, data sensed during the operation of the pacemaker may be stored in the memory MEM 42 for later retrieval and analysis. This includes atrioventricular interval data that are acquired by the control unit CTRL 40. Control unit CTRL 40 is adapted to determine the atrioventricular interval data, as required for automatic atrioventricular interval analysis, by determining the time interval between an atrial event, either sensed (As) or stimulated (Ap), and an immediately following ventricular sensed event Vs as indicated by the ventricular sensing stage V-SENS 38.

The pacemaker 10 further includes a telemetry circuit TEL 46 connected to the control unit

CTRL 40 by way of a suitable command/data bus. Telemetry circuit TEL 46 allows for wireless data exchange between the pacemaker 10 and a remote programming or analyzing device, which can be part of a centralized service center serving multiple pacemakers.

The pacemaker 10 in FIG. 1 is referred to as a dual chamber pacemaker because it interfaces with both the right atrium 26 and the right ventricle 28 of the heart 12. Those portions of the pacemaker 10 that interface with the right atrium, e.g., the lead 14, the P-wave sensing stage A-SENSE 36, the atrial stimulation pulse generator A-STIM 32 and corresponding portions of the control unit CTRL 40, are commonly referred to as the atrial channel. Similarly, those portions of the pacemaker 10 that interface with the right ventricle 28, e.g., the lead 16, the R-wave sensing stage V-SENSE 38, the ventricular stimulation pulse generator V-STIM 34, and corresponding portions of the control unit CTRL 40, are commonly referred to as the ventricular channel

In order to allow rate adaptive pacing in a DDDR or a DDIR mode, the pacemaker 10 further includes a physiological sensor ACT 48 connected to the control unit CTRL 40 of the pacemaker 10. While this sensor ACT 48 is illustrated in FIG. 2 as being included within the pacemaker 10, it should be understood that the sensor may also be external to the pacemaker 10, yet still be implanted within or carried by the patient.

Control unit CTRL 40 is adapted to put the pacemaker 10 into a VOO or a DOO mode of is operation wherein either only the ventricle or the ventricle and the atrium are stimulated with a fixed stimulation rate and no sensing nor any inhibition. For such a mode of operation, the ventricular stimulation pulse generator V-STIM 34, or both the ventricular stimulation pulse generator V-STIM 34 and the atrial stimulation pulse generator A-STIM 32, can be connected to a fixed rate oscillator that is insensitive to MRI magnetic fields.

The pacemaker 10 also includes a magnetic field detector MAG-DETEC 50 that is connected to control unit CTRL 40. The magnetic field detector MAG-DETEC 50 is adapted to generate a detection signal characteristic of the magnetic field used for magnetic resonance imaging (MRI). The control unit CTRL 40 is adapted to process the detection signal generated by the magnetic field detector MAG-DETEC 50 and to thus positively recognize the presence of a magnetic field as used for magnetic resonance imaging (MRI). The control unit CTRL 40 responds to detection of a presence of a magnetic field (as used for magnetic resonance imaging) by causing the implantable medical device to enter an MRI-safe mode of operation.

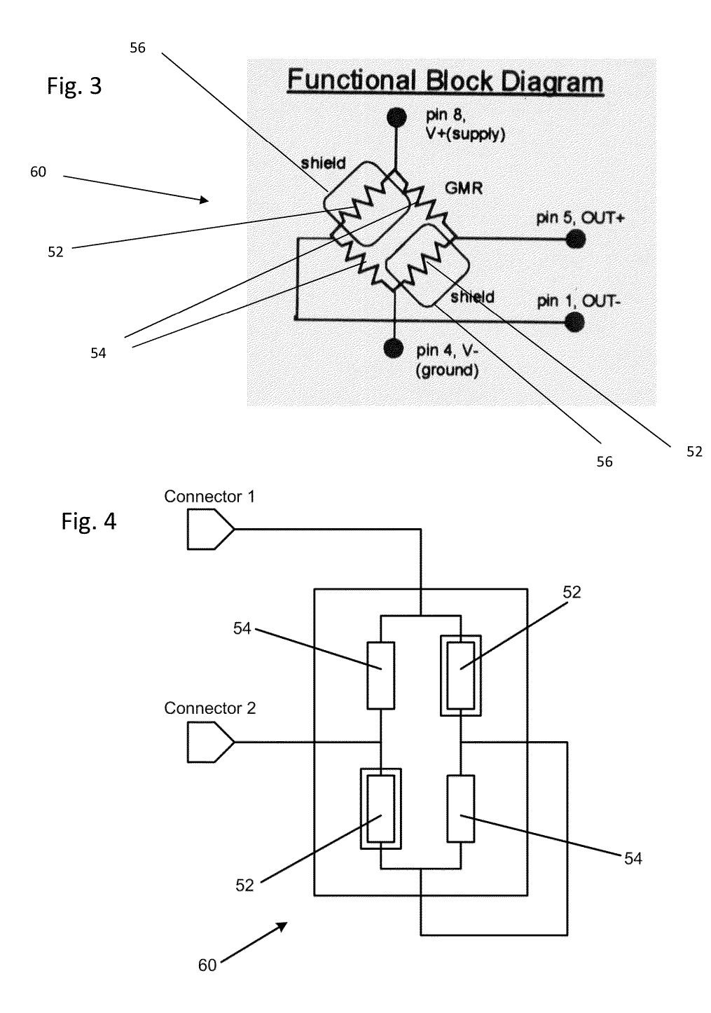

The magnetic field MAG-DETEC 50 includes a sensor arrangement of one or more giant magneto-resistive (GMR) sensing circuits as depicted in FIGS. 3-23.

When a magnetic field is applied, the GMR effect results in a decrease in the electrical resistance of a multilayer structure including alternating layers of ferromagnetic and paramagnetic thin films. Referring to FIG. 3, a GMR sensing circuit 60 typically includes four resistors 52 and 54 arranged in a Wheatstone bridge configuration. The four resistors are fabricated with the same resistance value using the same materials, e.g., using thin-film technology on a silicon substrate. Two resistors 52 have magnetic shields 56 placed over them, whereas the other two resistors 54 are unshielded and may have flux concentrators to concentrate the magnetic field into them. The resistors are typically within the range of 2 kΩ to 50 kΩ. Due to these low resistance values, special techniques are required to reduce the average power required by the GMR control and processing circuit for use in an implantable medical device. For example, reduction of the average power may be done by using a low operating voltage, or by “strobing” the GMR sensor with a low duty cycle.

The behavior of the GMR sensing circuit 60 of FIG. 3 depends on the strength of the magnetic field to which the GMR sensor is exposed. With no magnetic field applied and with a supply voltage applied between V− and V+, the output of the Wheatstone bridge (i.e., the voltage between OUT+ and OUT−) will be close to zero, although there may be a small offset due to the earth's magnetic field and/or resistor value mismatch. When a magnetic field is applied to the GMR sensing circuit 60 in the axis of sensitivity, the unshielded resistors will decrease in resistance as the magnetic field strength increases. The shielded resistors will also change in resistance, but by only a small amount compared to the change in the unshielded resistors over the operating range of the GMR sensing circuit 60 in the bridge configuration. This results in an increase in the voltage difference between OUT− and OUT+ when a voltage is applied between V− and V+. The change in resistance is independent of whether the field is N-S or S-N. The change in GMR resistance from that due to the background earth's magnetic field (approx. 0.05 mT) to magnetic saturation of the device results in a typical resistance change of 12% to 25%.

The GMR sensing circuit in FIG. 3 is sensitive to the axis of the magnetic field, as is also the case with a reed switch. The “window” where the orientation of the applied magnet axis results in the reed switch opening is similar to that of a GMR sensing circuit in a practical application.

GMR sensing circuits include GMR elements (electronic resistors) that change their resistance as a function of a magnetic field impinging on them. In general, the stronger the magnetic field, the lower the device's resistance.

A GMR element is sensitive to the orientation of the magnetic field with respect to the GMR element. Rotating a GMR element 90 degrees in a fixed magnetic field can result in a very significant change in the GMR element's resistance.

The change in resistance in a GMR element can be modified by shielding it with either a material that reduces the magnetic field impinging on the GMR element, or a with a material that increases the magnetic field impinging on the GMR element. Both of these is materials and approaches are referred to as “shielding” in this document.

GMR sensing circuits may be designed to detect or measure low level magnetic fields. These are typically an arrangement of four GMR elements in a bridge configuration, as in FIG. 3. Two opposite legs of the bridge are shielded and the other two are not. When the bridge is magnetically stimulated, the output differential voltage is proportional to the magnetic field impinging on it within a limited range, and within a limited orientation of the magnetic field and of the GMR elements 52 and 54 themselves.

When looking at the overall resistance of these devices for use in higher magnetic field detection circuits, such as for MRI static field detection, there is a significant difference in the resistance change of the shielded vs. the unshielded GMR elements with respect to the impinging field strength and with respect to the orientation of the magnetic shield of the GMR element.

In one orientation a GMR element can give better guaranteed detection above a certain limit, for example, 30 mT. But when rotated 90 degrees, it may provide very poor guaranteed non-detection below a lower limit, say 10 mT. Shielding of the GMR element can modify this behavior. By using a combination of shielded and unshielded GMR elements at different orientations, better detection of fields of certain strengths, and exclusion of fields of other strengths, can be achieved.

Because GMR elements are sensitive to the orientation of an impinging magnetic field, prior art GMR sensor arrangements may not be able to reliably detect both high and low magnetic fields with different orientations. Therefore, the magnetic field detector 50 of FIG. 2 includes a magnetic field sensor arrangement with a GMR sensing circuit that allows voltage measurement not only across the bridge (as in FIG. 3), but at further measurement points of the GMR sensing circuit. One way of selectively creating further measurement points is to selectively short one or more GMR elements. FIGS. 4-8 illustrate measurement configurations that allow further voltage measurements on a GMR sensing circuit similar to the one depicted in FIG. 3. Thus, several measurement configurations are made possible that allow various voltage measurements that can be evaluated both absolutely and in relation to each other.

As can be seen in FIG. 4, one measurement configuration is to measure the impedance between the terminals of a first unshielded GMR element (resistor 54) while the second unshielded GMR element (the other resistor 54) is shorted.

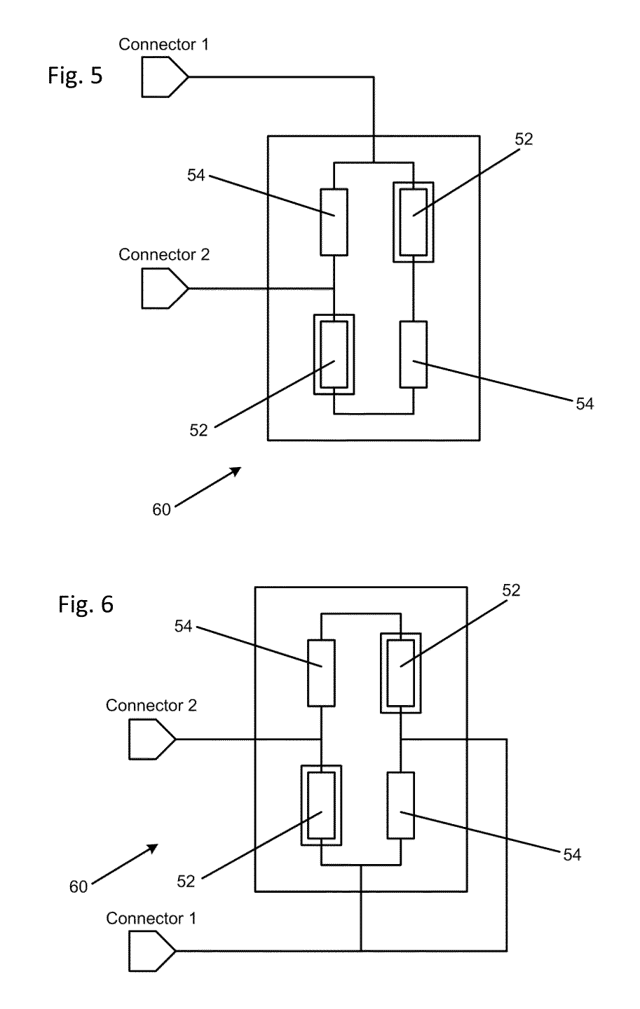

FIG. 5 depicts another measurement configuration that differs from the configuration in FIG. 4 in that the second unshielded GMR element 54 is not shorted.

FIG. 6 discloses yet another measurement arrangement wherein the second unshielded GMR element (resistor 54) is shorted and voltage measurement is carried out over a shielded GMR element 52 (shielded resistor 52).

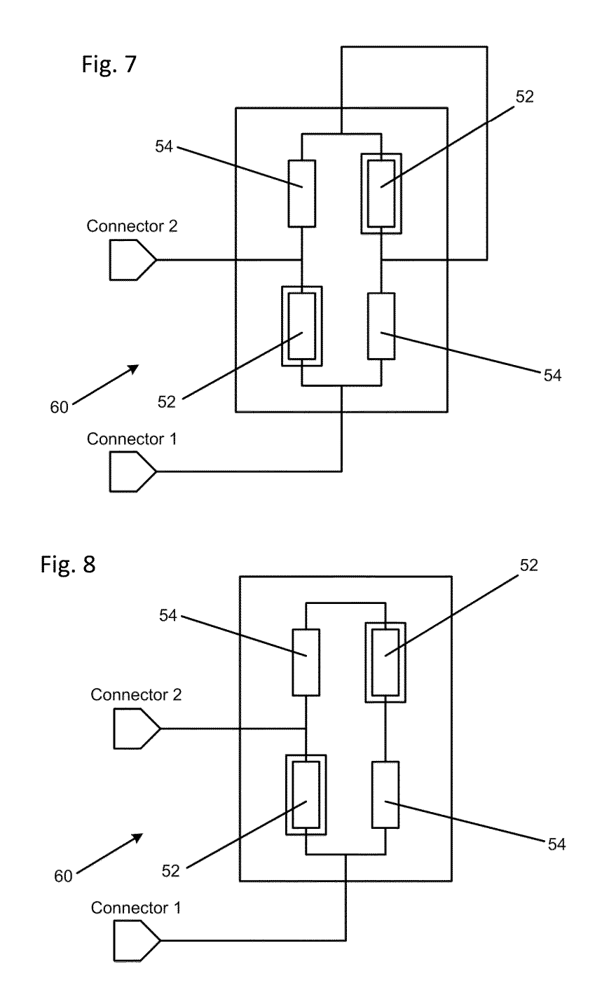

In FIG. 7, yet another measurement configuration is depicted wherein a voltage measurement is made across one shielded GMR element 52 (resistor 52) while the other shielded GMR element 52 (the other shielded resistor 52) is shorted.

FIG. 8 illustrates yet another measurement configuration wherein measurement is again carried out over one shielded GMR element 52 (shielded resistor 52) while the other shielded GMR element 52 is not shorted (as opposed to the arrangement in FIG. 6).

Thus, FIGS. 3-8 illustrate several of the voltage measurement configurations which provide for different measured voltages that can be evaluated in an absolute manner, and also in relation to each other.

Preferably, the evaluation circuit 64 (FIG. 2) is configured to provide a relative evaluation of voltages by generating differences between measured voltages.

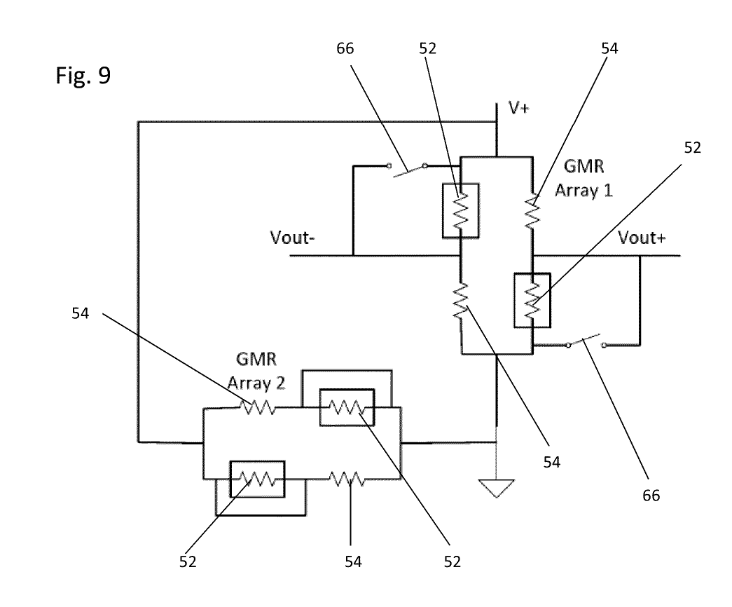

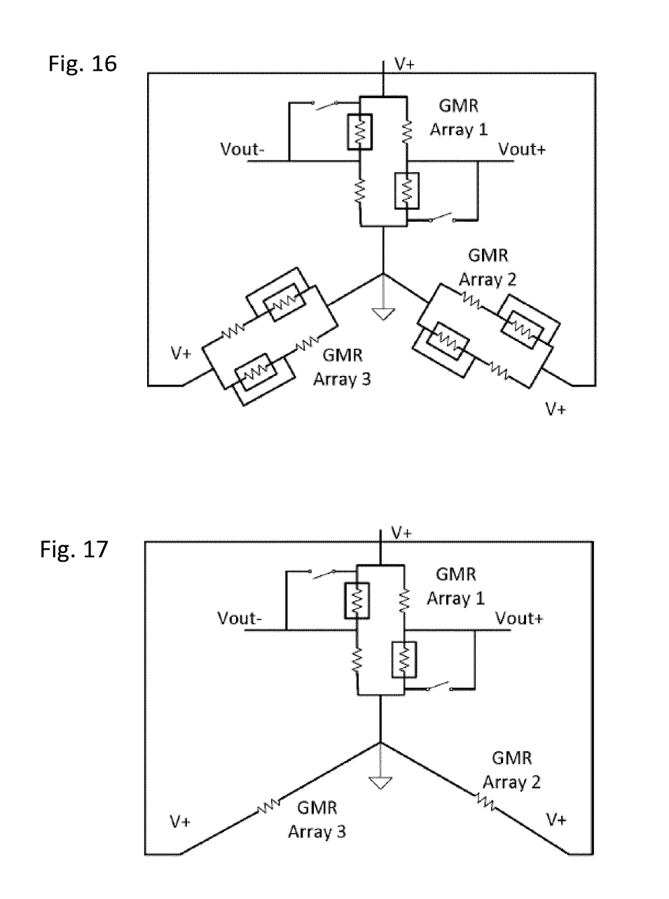

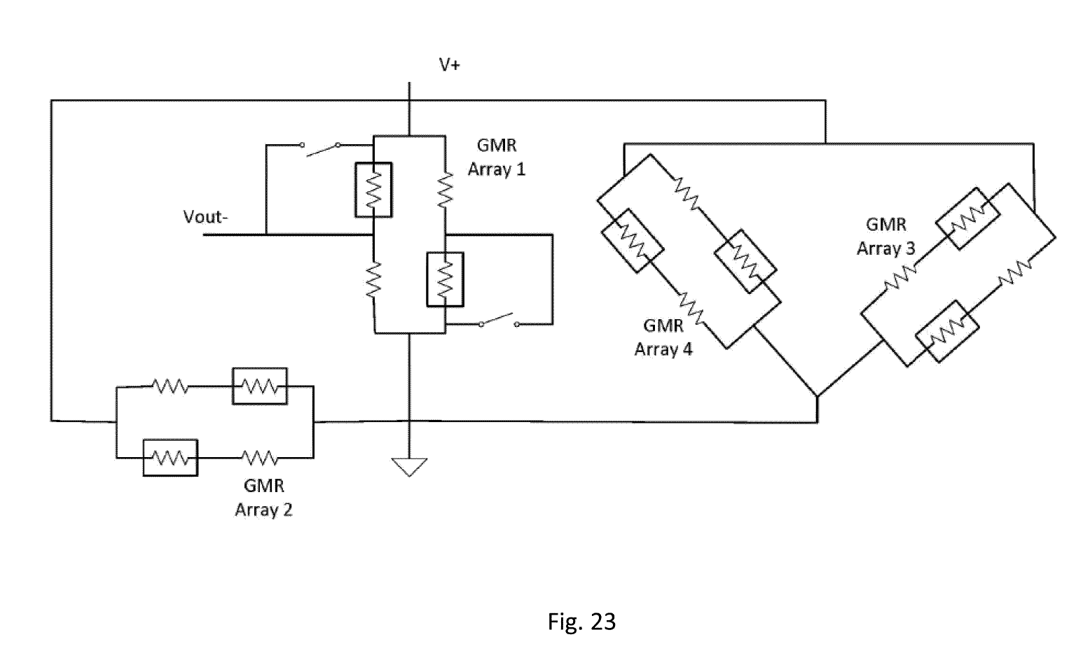

FIGS. 9-23 illustrate further examples of magnetic field sensor arrangements that include more than one GMR sensing circuit. In particular, FIGS. 9-15 illustrate magnetic field sensor arrangements including two GMR sensing circuits, and FIGS. 16-22 illustrate versions wherein the magnetic field sensor arrangement includes three GMR sensing circuits. Finally, FIG. 23 illustrates a version of a magnetic field sensor arrangement including four GMR sensing circuits.

FIGS. 9-23 are schematic representations wherein the GMR sensing circuits are shown at various angles to each other within each magnetic field sensor arrangement. Combining two or more GMR sensing circuits can decrease the sensitivity of the overall magnetic field sensor arrangement to the orientation of an impinging magnetic field. Thus, the magnetic field sensor arrangement can provide more uniform results, which depend on the strength of the impinging magnetic field rather than on the field's orientation.

In FIGS. 9-23 the individual GMR sensing circuits are designated as “GMR array”. The version depicted in FIG. 9 is a magnetic field sensor arrangement including two GMR sensing circuits.

The two GMR sensing circuits are oriented at 90 degrees relative to each other. Each GMR sensing circuit (GMR array 1 and GMR array 2) includes two unshielded GMR elements and two shielded GMR elements. In the second GMR sensing circuit (GMR array 2), the two shielded GMR elements are shorted and thus ineffective. The same effect could also be achieved with a GMR sensing circuit including only one unshielded GMR element.

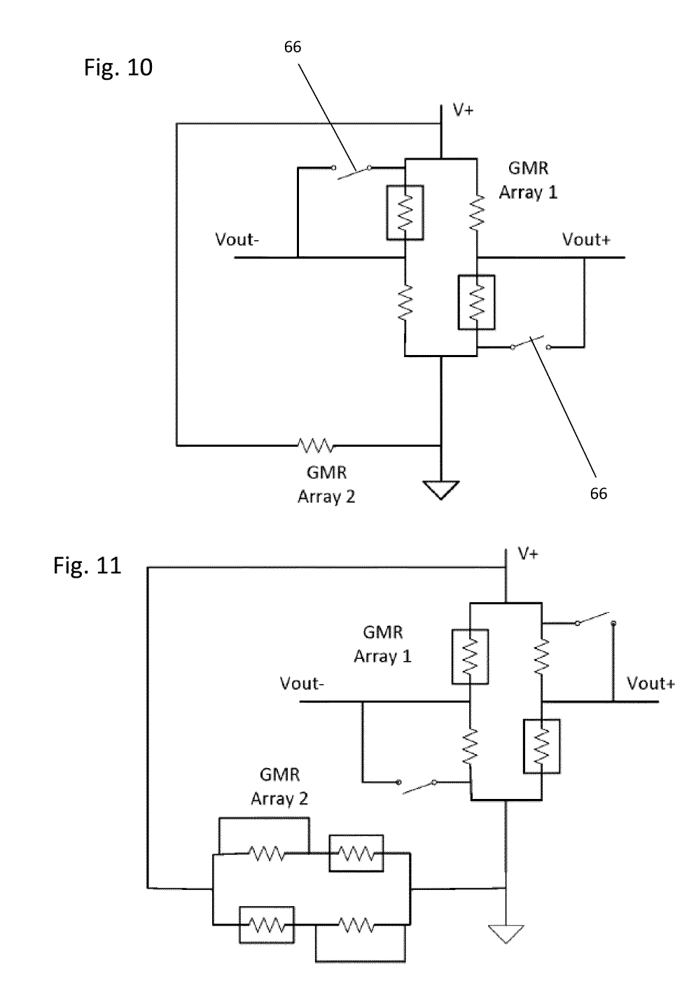

With respect to the first GMR sensing circuit (GMR array 1), the versions of FIGS. 9 and 10 are identical. Switches 66 the shielded GMR elements in the first GMR sensing circuit to be selectively shorted. By use of the switches 66, the first GMR sensing circuit (GMR array 1) may be configured to be used as a bridge (similar to the one shown in FIG. 3) or for bulk resistance measurement, depending on the application.

The version of FIG. 11 is similar to the version of FIG. 9, but the first GMR sensing circuit (GMR array 1) includes switches 66 allowing shorting of the unshielded GMR elements of the first GMR sensing circuit. Similarly, in the second GMR sensing circuit (GMR array 2) of the version of FIG. 11, both unshielded GMR sensing elements are shorted and thus ineffective.

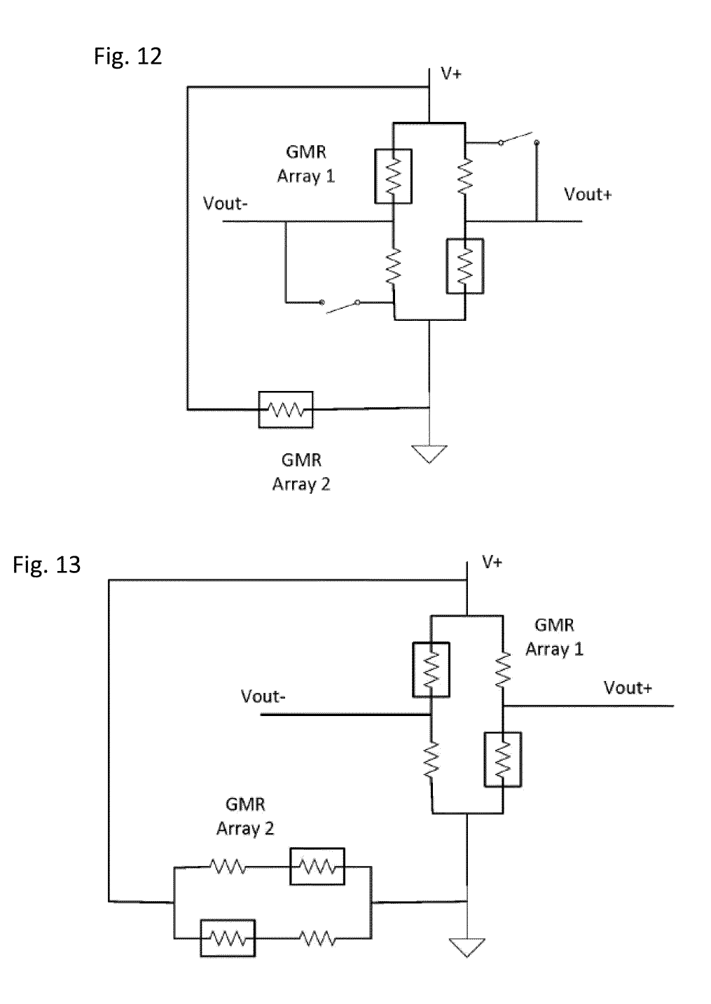

FIG. 12 depicts a magnetic field sensor arrangement that is equivalent to the version of FIG. 11. In the version of FIG. 12, both effective shielded GMR elements of the second GMR sensing circuit are replaced by a single shielded GMR element.

Again, the switches 66 in the versions of FIGS. 11 and 12 allow use of the first GMR sensing circuit (GMR array 1) as a bridge or for bulk resistance measurement, respectively, depending on whether or not switches 66 are open or closed, respectively.

FIG. 13 illustrates a version with two GMR sensing circuits (two GMR arrays) arranged at 90 degrees with respect to each other and with no shorted GMR elements. The first GMR sensing circuit (GMR array 1) is used as a traditional full bridge as in FIG. 3, whereas the second GMR sensing circuit (GMR array 2) is configured for bulk resistance measurement.

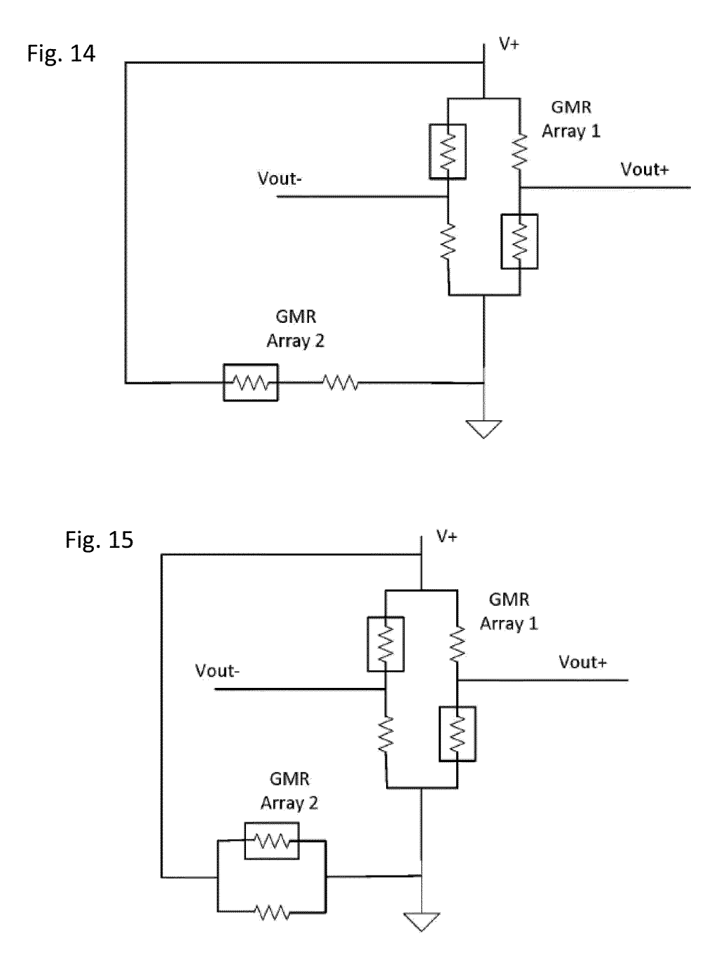

Again, the effect achieved by the version of FIG. 13 can be achieved with a more simple second GMR sensing circuit. FIG. 14 discloses a simplified second GMR sensing circuit equivalent to the second GMR sensing circuit in the version of FIG. 13, wherein the second GMR sensing circuit includes one shielded GMR element and one unshielded GMR element connected in series.

Alternatively, the effect of the second GMR sensing circuit of the version of FIG. 13 can be achieved with a second GMR sensing circuit including one shielded GMR element and one unshielded GMR element connected in parallel to each other, as in FIG. 15.

While the use of two GMR sensing circuits in combination with each other will reduce the magnetic field sensor arrangement's sensitivity with respect to the spatial orientation of an impinging magnetic field, this advantage comes at the costs of higher complexity and power drain. The versions of FIGS. 10, 12, 14 and 15 provide acceptable behavior at acceptable expense for most medical implant applications, whereas the versions of FIGS. 9 and 11 may not always be suitable.

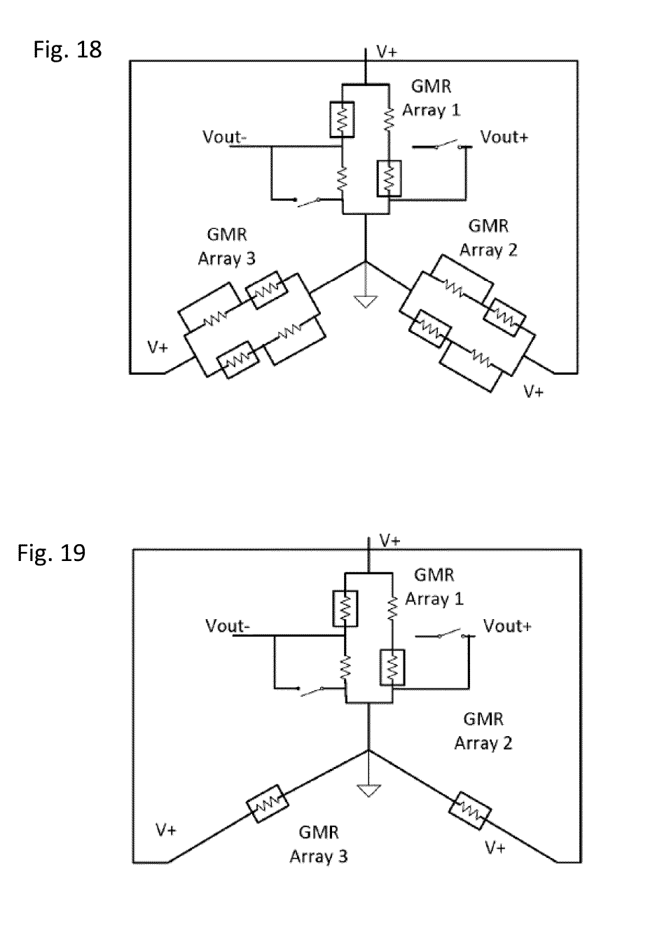

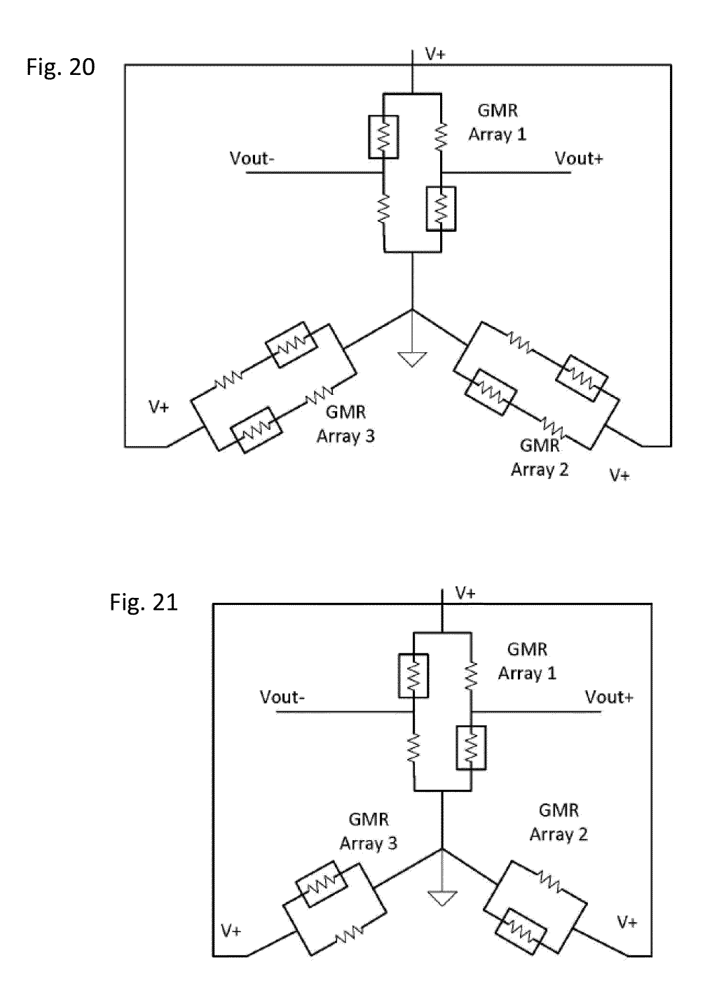

FIGS. 16-22 each disclose different versions of a magnetic field sensor arrangement including three GMR sensing circuits (three GMR arrays) with different spatial orientations. In particular, the three GMR sensing circuits of each of the versions of FIGS. 16-22 are spatially oriented at 0°, 60° and 120°. As in the versions of FIGS. 9-12, the first GMR sensing circuit (GMR array 1) includes switches 66 for selectively shorting one or more of the GMR elements, thereby allowing a voltage measurement that corresponds either to the traditional Wheatstone bridge arrangement or to a bulk voltage measurement. With respect to the second and the third GMR sensing circuits of the versions of FIGS. 16-22, these may either include shorted GMR elements (FIGS. 16 and 18), or they may be replaced by just a single GMR element (FIGS. 17 and 19), in a manner similar to the versions of FIGS. 10 and 12. Likewise, the magnetic field sensor arrangement may include no switches, as in the versions of FIGS. 20-22.

In the versions of FIGS. 20-22, the first GMR sensing circuit (GMR array 1) is connected in a traditional Wheatstone bridge arrangement similar to the version of FIG. 3 or of FIGS. 13-15.

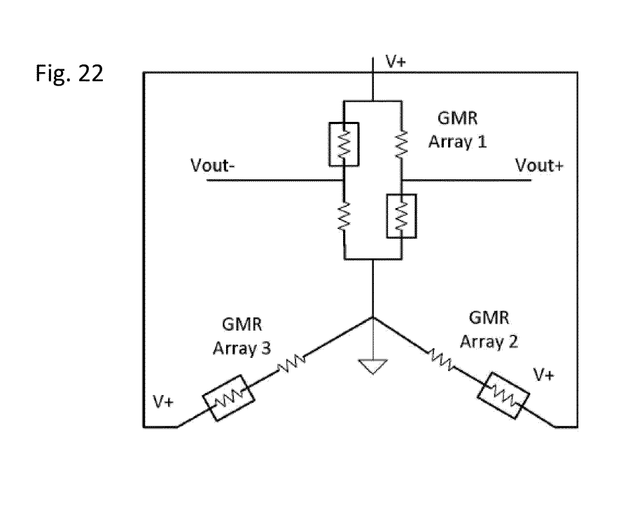

Further, none of the elements of GMR Array 1 is shorted in the versions of FIGS. 20-22. As can be seen in FIGS. 21 and 22, the second and the third GMR sensing circuits can be simplified and replaced by a GMR sensing circuit including only one shielded and one unshielded GMR element, similar to the versions of FIGS. 14 and 15.

The first GMR sensing circuit can be configured for use as a magnet detector or for an MRI field detector. The three GMR sensing circuits are arranged with equal angular spacing so that they together provide a much more uniform MRI response with respect to angle than a single GMR, and than two GMRs oriented at 90 degrees with respect to each other. It is preferred that the GMR sensing circuits be identical to better ensure behavior matching of the circuits, which is important when using the circuits in parallel.

Shorting out the shielded legs of a GMR sensing circuit (GMR array) yields a device that has high sensitivity at low fields and lower sensitivity at high fields. Shorting out the is unshielded legs provides a device with low sensitivity at low fields and moderate sensitivity at higher fields.

A magnetic field sensor arrangement including three GMR sensing circuits provides very good response at somewhat higher cost. A sensor arrangement can be constructed with any number of circuits arrays greater than 1, but the use of 2-4 arrays is typically more cost-efficient.

Even less sensitivity to magnetic field orientation can be achieved with a magnetic field sensor arrangement including four GMR sensing circuits, as in the version of FIG. 23. Here the four GMR sensing circuits are provided that are spatially oriented at 0 degrees, 45 degrees, 90 degrees and 145 degrees. One of the GMR sensing circuits (GMR array 1) includes switches for selectively shorting its shielded GMR elements. For each of the four GMR sensing circuits of the version of FIG. 23, the same operations are possible as discussed with respect to the versions of FIGS. 9 to 23.

Exemplary versions of the present invention have been shown and described, and it should be apparent to those of ordinary skill in the art that modifications to the invention may be made without departing from the spirit and scope of the present invention. The invention can readily be adapted to different kinds of applications in the field of magnetic field detection by following the teachings herein. The invention encompasses all changes, modifications and alterations that fall within the scope of the following claims.

What is claimed is:

1. A magnetic field sensor (50) including:
a. a giant magnetoresistance (GMR) sensing circuit (60) having GMR elements (52, 54) wherein:
(1) at least one first GMR element (52) is at least partially housed in either:
(a) a flux-reducing first shielding material that reduces the magnetic field impinging on the first GMR element (52), or
(b) a flux-concentrating first shielding material that amplifies the magnetic field impinging on the first GMR element (52);
(2) at least one second GMR element (54) is either:
(a) housed in a second shielding material which either:
i. amplifies the magnetic field impinging on the second GMR element (52) where the first shielding material reduces the magnetic field impinging on the first GMR element (52), or
ii. reduces the magnetic field impinging on the second GMR element (52) where the first shielding material amplifies the magnetic field impinging on the first GMR element (52); or
(b) not housed in a shielding material;
b. a measurement circuit (62) configured to measure voltages at different locations in the GMR sensing circuit (60), whereby electric impedances between the different locations can be determined to provide a signal that indicates strength of an external magnetic field.

2. The magnetic field sensor (50) of claim 1 wherein the GMR sensing circuit (60) is a bridge circuit including four GMR elements (52, 54).

3. The magnetic field sensor (50) of claim 1 wherein the GMR elements (52, 54) are resistors formed of giant magneto-resistive material.

4. The magnetic field sensor (50) of claim 1 including at least two of the GMR sensing circuit (60), wherein the GMR sensing circuits (60) have different spatial orientations.

5. The magnetic field sensor (50) of claim 4 wherein the GMR elements (52, 54) are situated in a common spatial plane.

6. The magnetic field sensor (50) of claim 1 wherein at least one of the GMR elements has a spatial orientation that differs from the orientation of at least one of the other GMR elements by a non-perpendicular angle of more than 0°.

7. The magnetic field sensor (50) of claim 6 wherein the GMR elements (52, 54) are situated in a common spatial plane.

8. The magnetic field sensor (50) of claim 1 further including means for electrically shorting at least one of the GMR elements (52, 54).

9. The magnetic field sensor (50) of claim 1 further including a switch, wherein closing the switch electrically shorts at least one of the GMR elements (52, 54).

10. The magnetic field sensor (50) of claim 1 further including an evaluation circuit (64) configured to evaluate both:
a. the determined impedances, and
b. relationships therebetween,
and thereby provide a signal that indicates strength of an external magnetic field.

11. The magnetic field sensor (50) of claim 10 wherein the evaluation circuit (64) is configured to evaluate the relationships between the determined impedances by determining the differences between the determined impedances.

12. The magnetic field sensor (50) of claim 1 provided within an implantable medical device.

13. The magnetic field sensor (50) of claim 12 wherein the implantable medical device is programmable and includes a processor operationally connected to the magnetic field sensor (50).

14. The magnetic field sensor (50) of claim 12 wherein the implantable medical device is an implantable pulse generator configured to generate stimulation pulses.

15. A magnetic field sensor (50) of claim 1 wherein each of the different locations at which the measurement circuit (62) measures voltages is spaced from each other location by at least one of the GMR elements (52, 54).

16. A method for determining the presence of a magnetic field using the magnetic field sensor (50) of claim 1, the method including:
a. determining the voltages between different locations of the GMR sensing circuit (60);
b. determining electric impedances between at least some of the different locations:
c. generating from the determined electric impedances a signal that indicates strength of an external magnetic field.

17. A magnetic field sensor (50) including:
a. a giant magnetoresistance (GMR) sensing circuit (60) having GMR elements (52, 54) wherein:
(1) at least one first GMR element (52) is at least partially housed in either:
(a) a flux-reducing first shielding material that reduces the magnetic field impinging on the first GMR element (52), or
(b) a flux-concentrating first shielding material that amplifies the magnetic field impinging on the first GMR element (52);
(2) at least one second GMR element (54) is either:
(a) housed in a second shielding material which either:
i. amplifies the magnetic field impinging on the second GMR element (52) where the first shielding material reduces the magnetic field impinging on the first GMR element (52), or
ii. reduces the magnetic field impinging on the second GMR element (52) where the first shielding material amplifies the magnetic field impinging on the first GMR element (52); or
(b) not housed in a shielding material;
b. an evaluation circuit (64) configured to provide a signal that indicates strength of an external magnetic field, the signal being dependent on voltages measured at different locations in the GMR sensing circuit (60), the different locations being spaced apart from each other by at least one of the GMR elements (52, 54).

18. The magnetic field sensor (50) of claim 16 wherein the GMR sensing circuit (60) is a bridge circuit.

19. The magnetic field sensor (50) of claim 17 further including a switch, wherein closing the switch electrically shorts at least one of the GMR elements (52, 54).

20. The magnetic field sensor (50) of claim 18 provided within an implantable medical device.
